(19) United States
(12) Patent Application Publication
(10) Pub. No.: US 2006/0241746 A1
(43) Pub. Date: Oct. 26, 2006

(54) Magnetic implants and methods for reshaping tissue

(76) Inventors: Emanuel Shaoulian, Newport Beach, CA (US); Shahram Moaddeb, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/111,682

(22) Filed: Apr. 21, 2005

(51) Int. Cl. A61F 2/24; A61N 2/10

(52) U.S. Cl. 623/2.37; 600/12

(57) ABSTRACT

Methods and devices for reshaping or reforming tissue, such as a mitral valve of a heart, are described. An implant includes a generally flexible body and a plurality of magnetic portions such that the magnetic portions interact to cause a change in the shape of the implant, which, in turn, effects a change in shape of the subject tissue. In one example, at least one implant is positioned within a coronary sinus to affect the shape of the mitral valve annulus. The implant may further include fixation mechanisms for securing the implant within a vessel and may provide for removability after desired deformation of the subject tissue has taken place.

### BACKGROUND OF THE INVENTION

1. Field of the Invention

The present invention generally relates to systems and methods to reshape tissue and, in particular, to dynamically reshape and resize the mitral valve annulus via implanting a magnetic device within the coronary sinus.

2. Description of the Related Art

In recent years, hundreds of thousands of individuals have undergone mitral valve replacement or repair. The mitral valve is a portion of the heart that is located between the chambers of the left atrium and the left ventricle. When the left ventricle contracts to pump blood throughout the body, the mitral valve closes to prevent the blood from being pumped back into the left atrium. In some individuals, whether due to genetic malformation, disease or injury, the mitral valve fails to close properly, causing a condition known as mitral regurgitation, whereby blood is pumped into the atrium upon each contraction of the heart muscle.

Mitral regurgitation is a serious, often rapidly deteriorating, condition that reduces circulatory efficiency. Oftentimes, mitral regurgitation is caused by geometric changes of the left ventricle, papillary muscles and mitral valve annulus. For example, certain diseases of the heart valves can result in dilation of the heart and one or more heart valves. When a heart valve annulus dilates, the valve leaflet geometry deforms and causes ineffective closure of the valve leaflets. The ineffective closure of the valve, or incomplete coaptation of the valve leaflets, can cause regurgitation of the blood, accumulation of blood in the heart and other problems.

Two of the more common techniques for restoring the function of a damaged mitral valve are valve replacement surgery and annuloplasty. In valve replacement surgery, the damaged leaflets are surgically excised, and the mitral valve annulus is sculpted to receive a replacement mechanical valve. In annuloplasty, the effective size of the valve annulus is contracted by attaching a prosthetic annuloplasty repair segment or ring to an interior wall of the heart around the valve annulus. The annuloplasty ring reinforces the functional changes that occur during the cardiac cycle to improve coaptation and valve integrity. Thus, annuloplasty rings help reduce reverse flow or regurgitation while permitting good hemodynamics during forward flow.

Each of these procedures, however, is highly invasive because access to the heart is obtained through an opening in the patient's chest, with the heart being bypassed to a heart-lung machine throughout the procedure. Most patients with mitral valve regurgitation, however, are often relatively frail, thereby increasing the risks associated with such an operation.

In response to the foregoing drawbacks, less invasive approaches have been proposed for aiding the closure of the mitral valve. These procedures involve the percutaneous placement of a manually-adjustable support structure in the coronary sinus close to the posterior leaflet of the mitral valve. The support structure is designed to push the vessel and surrounding tissue toward the anterior wall of the valve to aid its closure and to improve leaflet coaptation. This procedure, however, has several drawbacks. For example, the support structure does not allow for non-invasive alteration or adjustment and is oftentimes permanently implanted within the patient. Furthermore, a surgeon is unable to reduce the force of the support structure to reduce risk of artery pinching and is further unable to readjust the shape and size post-implant or during the implantation.

### SUMMARY OF THE INVENTION

In view of the foregoing, conventional systems and methods for treating valvular insufficiency do not provide for a less invasive approach that reduces strain on the patient. A need, therefore, remains for methods and devices that allow for non-invasive adjustment of an implant usable to treat valvular insufficiency and, in particular, mitral valvular insufficiency.

In one embodiment, a method is disclosed for changing a dimension of a mitral valve annulus of a heart. The method includes: positioning an implant at least partially in a coronary sinus of the heart, the implant comprising a first magnetic portion and a second portion. The second portion is responsive to a magnetic field emanating from the first magnetic portion, and the first magnetic portion and the second portion are configured to change the implant from a first configuration to a second configuration, which second configuration produces a change in the dimension of the mitral valve annulus.

In another embodiment, a tissue shaping device is disclosed. The tissue shaping device includes: an elongate, flexible body configured to fit within a coronary sinus of a heart; a first magnetic portion located in or on the body; and a second portion located in or on the body, the second portion being responsive to a magnetic field emanating from the first magnetic portion. Furthermore, the first magnetic portion is configured to interact with the second portion such that the body changes shape from a first configuration to a second configuration.

In another embodiment, a device for reshaping or reforming body tissue is disclosed. The device includes means for emanating a magnetic field; means for interacting with the means for emanating by responding to the magnetic field; and elongate, flexible means coupled to the means for emanating and the means for interacting, the elongate, flexible means configured to fit within a coronary sinus of a heart, and wherein the elongate, flexible means changes from a first configuration to a second configuration while the means for interacting responds to the magnetic field.

For purposes of summarizing the invention, certain aspects, advantages and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 illustrates a schematic view of a tissue shaping device positioned within a coronary sinus of a heart according to one embodiment of the invention.

FIGS. 2A and 2B illustrate perspective schematic views of a partial section of the heart including a mitral valve and a coronary sinus with an exemplifying embodiment of a tissue shaping device positioned therein.

FIG. 3 illustrates a perspective schematic view of a partial section of the heart including a mitral valve and a coronary sinus with another exemplifying embodiment of a tissue shaping device positioned therein.

FIG. 4 illustrates a perspective schematic view of a partial section of the heart including a mitral valve and a coronary sinus with another exemplifying embodiment of a tissue shaping device positioned therein.

FIG. 5 illustrates a perspective schematic view of a partial section of the heart including a mitral valve and a coronary sinus with multiple tissue shaping devices positioned therein according to one embodiment of the invention.

FIG. 6A illustrates a side schematic view of a tissue shaping device having an outer layer according to one embodiment of the invention.

FIG. 6B illustrates a transverse cross-sectional view of the tissue shaping device of FIG. 6A.

FIG. 6C illustrates a side schematic view of a tissue shaping device having an outer layer according to another embodiment of the invention.

FIG. 7A illustrates a side schematic view of an exemplifying embodiment of a tissue shaping device having securing fins.

FIG. 7B illustrates a side schematic view of an exemplifying embodiment of a tissue shaping device having securing tines.

FIG. 8A illustrates a side view of an exemplifying embodiment of a tissue shaping device having a curvilinear body.

FIG. 8B illustrates a side view of an exemplifying embodiment of a tissue shaping device having an elongated helical body.

FIGS. 9A-9C illustrate an exemplifying embodiment of a method for deploying a tissue shaping device within a coronary sinus.

### DETAILED DESCRIPTION OF THE PREFERRED EMBODIMENTS

The features of the systems and methods will now be described with reference to the drawings summarized above. Throughout the drawings, reference numbers are re-used to indicate correspondence between referenced elements. The drawings, associated descriptions, and specific implementation are provided to illustrate embodiments of the invention and not to limit the scope of the invention.

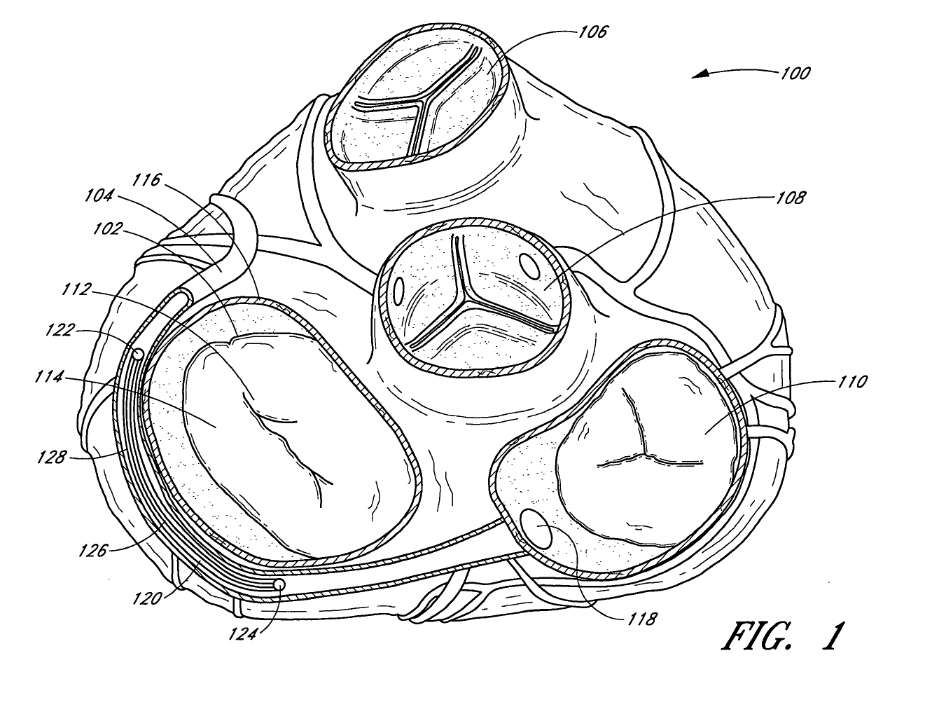

FIG. 1 illustrates a human heart 100 with the atria removed to expose a mitral (left atrioventricular) valve 102 and a coronary sinus 104. Also generally shown in FIG. 1 are a pulmonary valve 106, an aortic valve 108, and a tricuspid valve 110 of the heart 100.

The mitral valve 102 includes an anterior (aortic) leaflet 112, a posterior leaflet 114 and an annulus 116. When healthy, the annulus 116 encircles the leaflets 112, 114 and maintains their spacing to provide closure during left ventricular contraction. The coronary sinus 104 partially encircles the mitral valve 102 substantially adjacent to the mitral valve annulus 116 and extends from an ostium 118, or opening to the right atrium, to the anterior interventricular (“AIV”) sulcus or groove. In general, the coronary sinus 104 is located within the same plane as the mitral valve annulus 116, which makes the coronary sinus 104 available for placement therein of a tissue shaping device 120.

With reference to the embodiment depicted in FIG. 1, the tissue shaping device 120 includes magnetic portions, including a distal end 122 and a proximal end 124, connected by an elongated body 126. The tissue shaping device 120 further comprises an outer jacket 128 that encapsulates the elongated body 126 and the ends 122, 124. In one embodiment, the tissue shaping device 120 is a dynamically adjustable device usable to reshape or resize the mitral valve annulus 116 according to the needs of the patient. In particular, the tissue shaping device 120 is advantageously capable of affecting the shape of the coronary sinus 104, which, in turn, affects the shape of the mitral valve annulus 116. For example, the tissue shaping device 120 may be used to cause a change in at least one dimension of the mitral valve annulus 116. Appropriately affecting the shape of the mitral valve annulus 116 aids closure of the leaflets 112, 114 to improve coaptation, thereby correcting mitral valve insufficiency.

In one embodiment, the ends 122, 124 of the tissue shaping device 120 comprise a magnetic material. As illustrated, the ends 122, 124 are generally spherical in shape. In other embodiments, either or both of the ends 122, 124 may be in the shape of a rod, a disc, a cube or the like. In certain embodiments, the magnetic ends 122, 124 advantageously comprise a ferromagnetic material.

The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the heart 100.

Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (Neodynium Iron Boron), SmCo (Samarium Cobalt), ferrite and/or AlNiCo (Aluminum Nickel Cobalt) particles. The biocompatible polymer can comprise, for example, polycarbonate, silicone rubber, polyurethane, silicone elastomer, a flexible or semi-rigid plastic, combinations of the same and the like.

In certain preferred embodiments, at least one of the magnetic ends 122, 124 includes one or more rare-earth elements or rare-earth alloys, such as, for example, alloys of NdFeB, SmCo, AlNiCo, combinations of the same and the like.

In one embodiment, each of the ends 122, 124 has a diameter or thickness of approximately 2 to 4 mm, which facilitates placement and/or removal of the tissue shaping device 120 in or from the coronary sinus 104.

Although disclosed with reference to particular embodiments, the tissue shaping device 120 may include a wide variety of alternative forms, shapes and/or positions for magnetic portions instead of, or in combination with, at least one of the ends 122, 124. For example, the tissue shaping device 120 may include at least one magnetic portion that is located along the body 126 instead of at one of the ends of the body 126. In one embodiment, the magnetic portion is located near one of the ends of the body 126. In yet other embodiments, the tissue shaping device 120 may include more than two magnetic portions located along the body 126.

In yet other embodiments, the tissue shaping device 120 may include at least one permanent magnet, such as a rare-earth alloy, and at least one generally unmagnetized ferromagnetic portion that responds to the magnetic field emanated by the permanent magnet(s). For example, in one embodiment, the end 122 may comprise a permanent magnet, and the end 124 may comprise a ferromagnetic metal or alloy.

In yet other embodiments, the tissue shaping device 120 includes at least one electromagnet. In such an embodiment, an electromagnetic transmitter, such as a resistive coil, may be used to activate the electromagnet(s). The transmitter may advantageously be located outside the coronary sinus 104 and/or the heart 100 and usable to non-invasively adjust the shape of the tissue shaping device 120 after the tissue shaping device 120 has been positioned within the coronary sinus 104.

In yet further embodiments, the tissue shaping device 120 may comprise at least one magnetic structure including a magnet comprising a hard ferromagnetic material and a magnetic flux shield comprising a soft ferromagnetic material overlaying at least a portion of the magnet. The flux shield may be used to focus and enhance the magnetic field of the magnet in a direction that the shield does not overlay (e.g., in the direction of another magnet positioned near the opposite end of the tissue shaping device 120).

In general, ends 122, 124 interact with each other to cause a change in the shape of the tissue shaping device 120, which, as discussed above, effects a change in the shape of the mitral valve annulus 116. In one embodiment, the interaction is a magnetic interaction that causes attraction (e.g., between poles of different polarity) and/or repulsion (e.g., between poles of like polarity) between the ends 122, 124. In one embodiment, the length and/or shape of the elongated body 126 connecting the ends 122, 124 is used to control the spacing between the ends 122, 124. This spacing, in turn, affects the magnetic field(s) between the ends 122, 124 and, in turn, affects the magnitude of the change in shape of the tissue shaping device 120.

The elongated body 126 comprises a flexible material, such as, for example, silicone rubber. Such a flexible material allows for appropriate bending or deformation of the tissue shaping device 120 to effect changes in the mitral valve 102. As illustrated, the elongated body 126 has a general rod-like shape. In other embodiments of the invention, the elongated body 126 may have different shapes or forms, such as, for example, a helical shape, an arcuate shape, an S-shape, a ribbon-like shape, a curvilinear shape, a braided-wire, multiple wires, combinations of the same or the like. Furthermore, the elongated body 126 may comprise other flexible materials in addition to or in place of silicone rubber, such as, for example, polyurethane; TEFLON®; a flexible non-magnetic material such as nitinol, platinum iridium (Pt/Ir), titanium, or tantalum; a composite of a polymer and a non-magnetic metal or alloy, combinations of the same or the like.

In yet other embodiments, at least one of the ends 122, 124, or other magnetic portions, may be integrated into the body 126. In such embodiments, the tissue shaping device 120 may comprise a rod-like shape.

The illustrated tissue shaping device 120 further includes the outer jacket 128. The outer jacket 128 advantageously encapsulates at least a portion of the elongated body 126 and/or ends 122, 124 such that they do not contact tissue or fluid of the patient. For example, the outer jacket 128 may advantageously prevent rare-earth alloys in the ends 122, 124 from direct exposure or contact with the patient. In one embodiment, the outer jacket 128 comprises a biocompatible, flexible material. For example, the outer jacket 128 may advantageously comprise a polyurethane tube. In other embodiments, the outer jacket 128 may comprise polytetrafluoroethylene (“TEFLON®”) or expanded polytetrafluoroethylene (ePTFE). In yet other embodiments, the biocompatible outer jacket 128 may comprise DACRON®, woven velour, heparin-coated fabric, bovine or equine pericardium, homograft, patient graft, cell-seeded tissue, combinations of the same or the like.

In yet another embodiment, the outer jacket 128 comprises a biodegradable jacket or sleeve that facilities removal of the tissue shaping device 120 from the coronary sinus 104. For example, once physical remodeling of the mitral valve 102 has taken place (as determined, for example, by viewing Doppler enhanced echocardiograms), which may generally occur within six to twelve months post-implant, the tissue shaping device 120 may be removed while the outer jacket 128 remains within the coronary sinus 104. In one embodiment, the biodegradable jacket advantageously comprises a polylactic acid (PLA). In other embodiments, the biodegradable jacket comprises poly vinyl alcohol (PVA) or the like. In yet other embodiments, the outer jacket 128 comprises multiple layers, such as, for example, a biocompatible inner layer and a biodegradable outer layer.

The tissue shaping device 120 is advantageously sized to fit within the desired vessel or tissue. With reference to FIG. 1, the tissue shaping device 120 is of a size that allows for insertion in or removal from the coronary sinus 104, such as through the use of an elongate tubular body (e.g., a catheter). In one embodiment, the tissue shaping device 120 has a length between approximately 4 mm and 150 mm. In a preferred embodiment, the tissue shaping device 120 has a length of approximately 50 mm. The tissue shaping device 120, in one embodiment, has a diameter of approximately 2 to 6 mm. In a preferred embodiment, the tissue shaping device 120 has a diameter of approximately 5 mm.

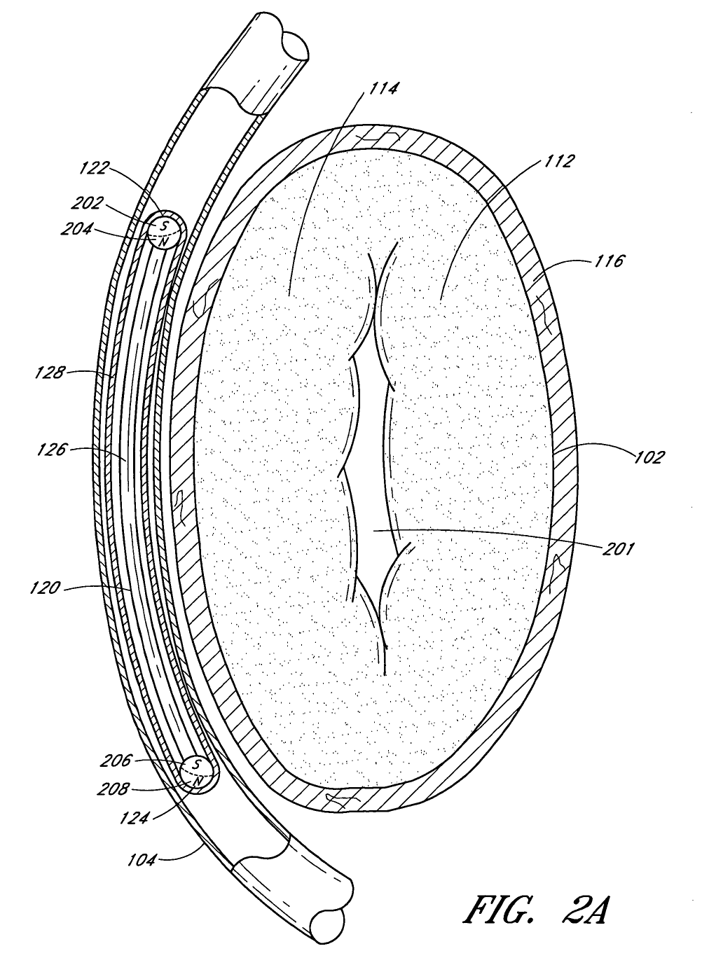

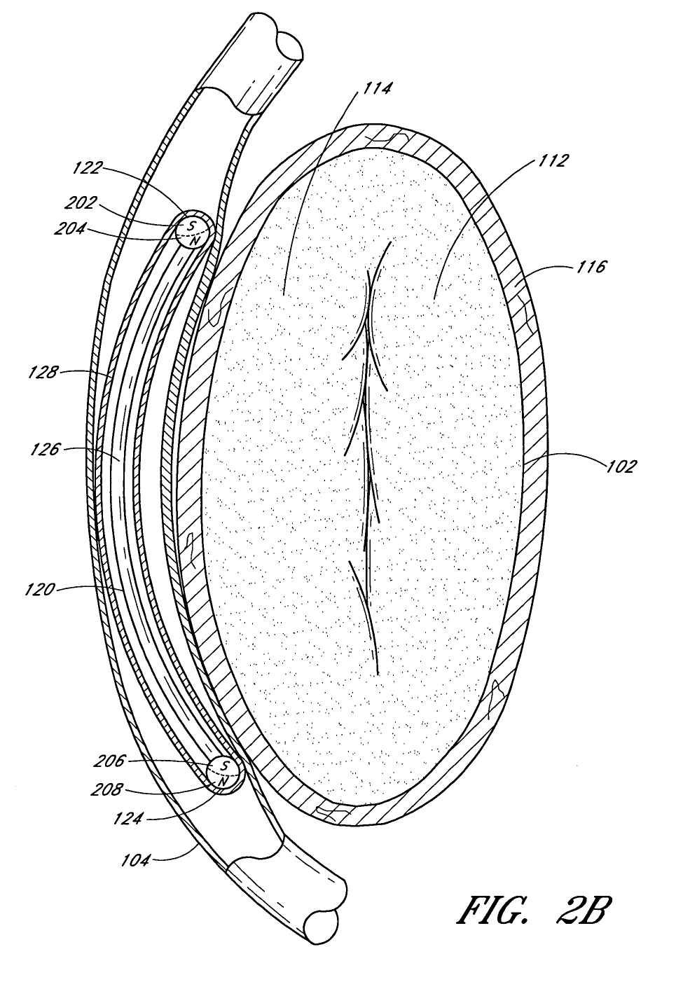

FIG. 2A illustrates an embodiment of the tissue shaping device 120, wherein the ends 122, 124 comprise permanent bipolar, or dipole, magnets. For simplification purposes, FIG. 2A illustrates only the mitral valve 102 and the coronary sinus 104 of the heart 100. As illustrated, a gap 201 exists between the leaflets 112, 114 due to insufficient closure of the leaflets 112, 114. As discussed above, this insufficient closure of the leaflets 112, 114 of the mitral valve 102 may be due to incomplete coaptation of the valve leaflets 112, 114 and can cause regurgitation of the blood, accumulation of blood in the heart and other potential health concerns.

As further shown in FIG. 2A, the ends 122, 124 of the tissue shaping device 120 are aligned to attract each other. In particular, the distal end 122 has a first north pole (N) 202 generally angled toward the posterior leaflet 114 at an acute angle with respect to the elongated body 126. In one embodiment, the first north pole 202 is aligned at an angle of between approximately thirty and sixty degrees with respect to the elongated body 126. The distal end 122 further comprises a first south pole (S) 204 generally aligned in an opposite direction of the first north pole 202.

The proximal end 124 has a second south pole (S) 206 generally angled toward the posterior leaflet 114 at an acute angle with respect to the elongated body 126. In one embodiment, the second south pole 206 is aligned at an angle of between approximately thirty and sixty degrees with respect to the elongated body 126. The proximal end 124 further includes a second north pole (N) 208 generally aligned in an opposite direction of the second south pole 206. In other embodiments, one or both of the first north pole 202 and the second south pole 206 may be aligned, with respect to the elongated body 126, at an angle between 0 and 90 degrees. In more preferred embodiments, one or both of the first north pole 202 and the second south pole 206 are aligned, with respect to the elongated body 126, at an angle between 45 and 90 degrees.

In one embodiment of the invention, the first north pole 202 of the distal end 122 and the second south pole 206 of the proximal end 124 attract each other due to their magnetic fields, thereby causing a slight bending of the tissue shaping device 120. In particular, the ends 122, 124 move generally toward the mitral valve 102, which causes the flexible elongated body 126 and the outer jacket 128 of the tissue shaping device 120 to take on a substantially arcuate shape.

As shown in FIG. 2B, as the tissue shaping device 120 changes shape, the tissue shaping device 120 contacts and pushes against the wall of the coronary sinus 104. This pressure causes a section of the coronary sinus 104 to straighten or to bend toward the mitral valve 102. This deformation of the coronary sinus 104 exerts pressure on the nearby mitral valve annulus 116 and causes a modification of the shape of the mitral valve 102. In particular, the deformation of the tissue shaping device 120 advantageously moves the posterior leaflet 114 of the mitral valve 102 toward the anterior leaflet 112 to lessen the gap 201 and to facilitate greater coaptation.

Precise deformation of the tissue shaping device 120 may be controlled through several factors. In one embodiment, the angling and/or magnetic strength of at least one of the ends 122, 124 may be selected to increase or decrease the amount of bending of the tissue shaping device 120. For example, increasing the magnetic strength of at least one of the ends 122, 124 will generally cause greater bending of the tissue shaping device 120 and, therefore, a greater pressure on the mitral valve annulus 116. In addition, advantageously angling at least one of the first north pole 202 and the second south pole 206 toward each other (i.e., toward the middle of the tissue shaping device 120) may cause an increased bending of the tissue shaping device 120. In another embodiment, the rigidity, shape and/or length of the elongated body 126 may be modified to increase or decrease the amount of deformation of the tissue shaping device 120.

Furthermore, in another embodiment of the invention, the tissue shaping device 120 may be initially deployed, within the coronary sinus 104, having a slight arcuate shape. Such an embodiment may facilitate an increased bending during and/or after deployment of the tissue shaping device 120 such that the tissue shaping device 120 assumes a more pronounced arcuate shape.

In one embodiment, the tissue shaping device 120 causes a pressure or force of approximately 2.22 newtons (0.5 pound-force) to approximately 13.34 newtons (3.0 pound-force) of displacement on the wall of the coronary sinus 104 in order to change at least one dimension of the mitral valve 102. Such pressure may cause the posterior leaflet 104 to move a distance of between approximately 5 mm and approximately 15 mm toward the anterior leaflet 112. In other embodiments, the posterior leaflet 114 moves a distance between approximately 2 mm and approximately 30 mm toward the anterior leaflet 112.

FIG. 3 depicts another exemplifying embodiment of the tissue shaping device 120 that forms an arcuate shape to cause a section of the wall of the coronary sinus 104 to push outward in the general direction of the mitral valve annulus 116. In particular, the ends 122, 124 attract such that a convex portion or side of the tissue shaping device 120 bows toward the mitral valve 102, which causes movement of the posterior leaflet 114 toward the anterior leaflet 112 to facilitate greater coaptation. As shown in FIG. 3, the first north pole 202 and the second south pole 206 are aligned to generally face each other. The phantom (broken) line depicted in FIG. 3 illustrates the shape of the tissue shaping device 120 prior to deformation (e.g., pre-implant), which deformation may be caused, in one embodiment, by magnetic attraction of the ends 122, 124.

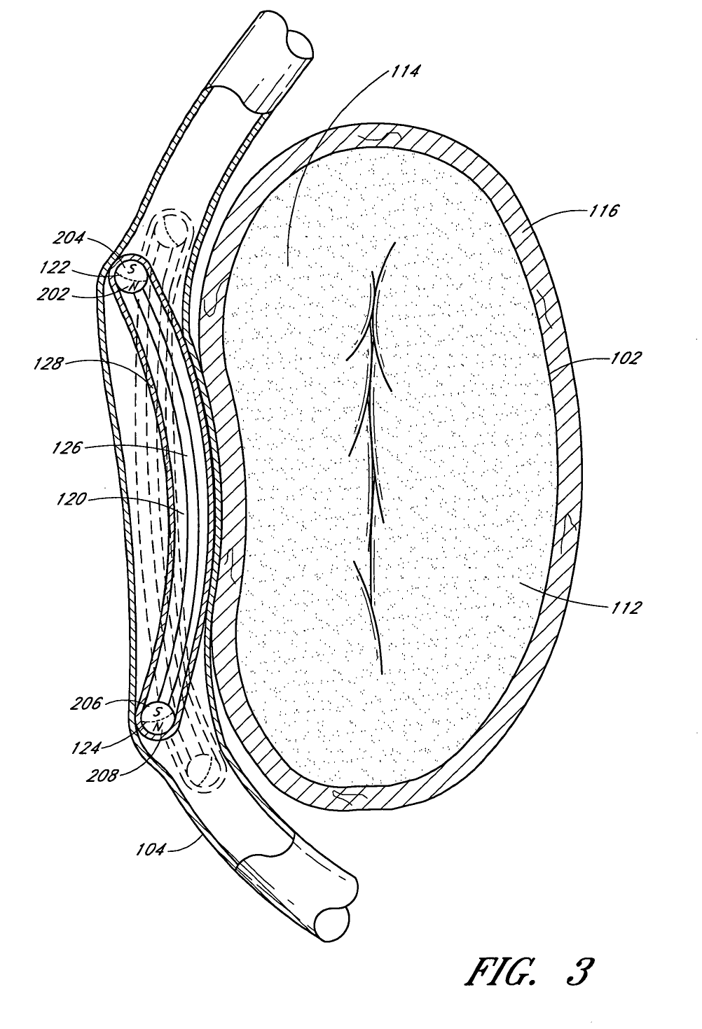

FIG. 4 depicts an embodiment of the invention wherein the tissue shaping device 120 includes ends 122, 124 configured to repel each other. As illustrated, the poles 202, 204 and the poles 206, 208 are generally oriented perpendicular to a general axis of the elongated body 126. In such a configuration, the ends 122, 124 repel each other and cause the elongated body 126 to straighten. In one embodiment, the elongated body 126 is advantageously arcuately shaped when initially deployed within the coronary sinus 104, as is shown by the phantom lines. As the ends 122, 124 repel each other, the straightening of the tissue shaping device 120 causes a corresponding straightening of a section of the coronary sinus 104. This, in turn, causes the outside wall of the coronary sinus 104 to engage the mitral valve annulus 116 such that the posterior leaflet 114 of the mitral valve 102 moves toward the anterior leaflet 112 to facilitate greater coaptation.

Although the foregoing embodiments have described the tissue shaping device 120 being generally used to reshape or resize a mitral valve of a human heart, the tissue shaping device 120 may be used with a wide variety of other valves, vessels, and/or tissue that require reshaping or reforming. For example, the tissue shaping device 120 may be used with other cardiac valves, such as, for example, the tricuspid valve, the pulmonary valve, or the aortic valve. In yet other embodiments, the tissue shaping device 120 may be used to reshape or reform left or right ventricles, gastric system tissue and/or organs (e.g., stomach), or the like.

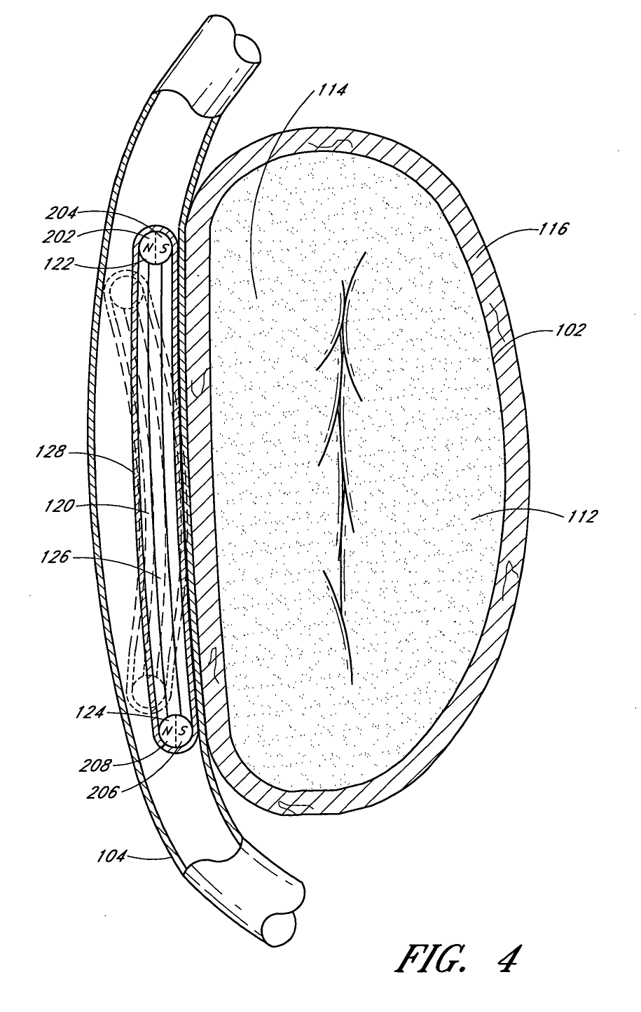

FIG. 5 illustrates an embodiment of the invention that provides for multiple implants having differing strengths or effects on the mitral valve 102. In particular, a first tissue shaping device 502 and a second tissue shaping device 504 are positioned within the coronary sinus 104. In one embodiment, the first tissue shaping device 502 exerts on the coronary sinus 104 a lower pressure or force than the second tissue shaping device 504 such that the second tissue shaping device 504 is capable of causing a greater reshaping of the mitral valve 102.

For example, in one embodiment, the first tissue shaping device 502 has a configuration similar to the tissue shaping device 120 depicted in FIG. 4 and includes lower-strength magnets. The second tissue shaping device 504 may have a configuration similar to the tissue shaping device 120 depicted in FIG. 3 and include higher-strength magnets compared to those of the first tissue shaping device 502. As illustrated, the first tissue shaping device 502 and second tissue shaping device 504 may be positioned in different locations along the length of the coronary sinus 104. In other embodiments, the tissue shaping devices 502, 504 may be positioned side-by-side in a parallel configuration to effect corresponding changes in the mitral valve 102. In yet other embodiments, more than two tissue shaping devices may be used, or the tissue shaping devices 502, 504 may be of different lengths, different shapes, or otherwise modified to provide for variable forces upon the coronary sinus 104 and the mitral valve annulus 116.

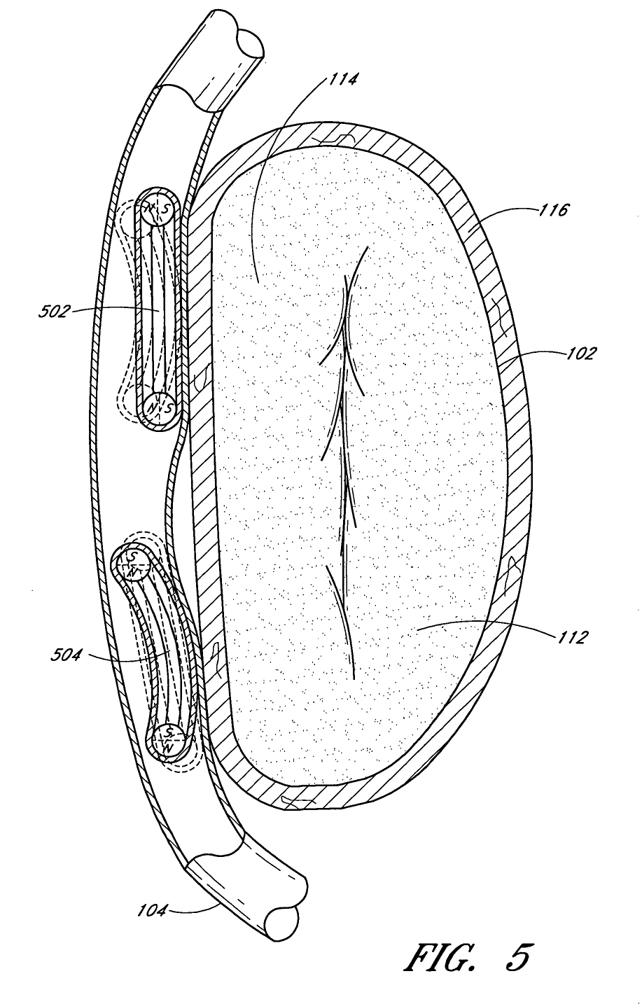

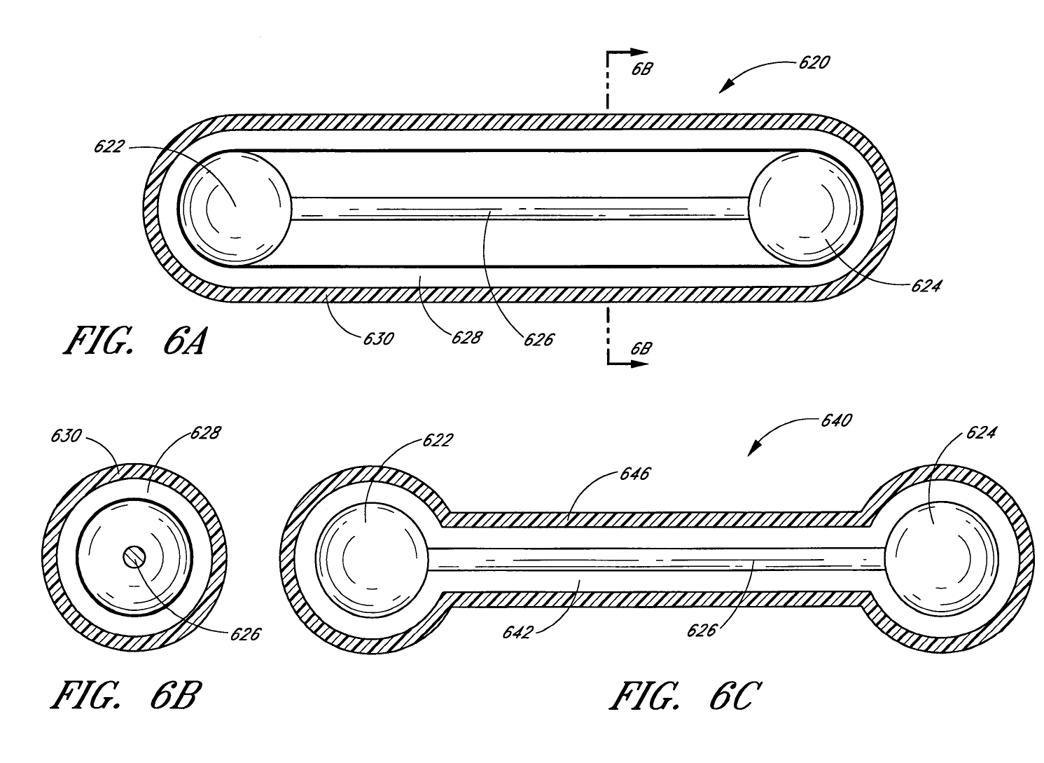

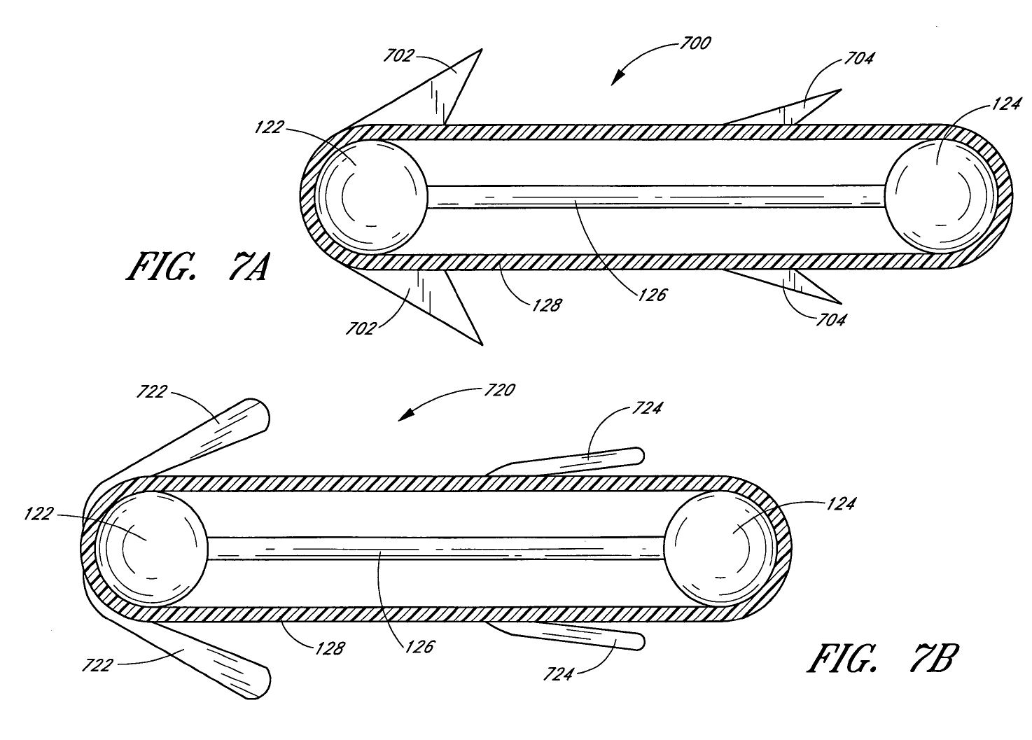

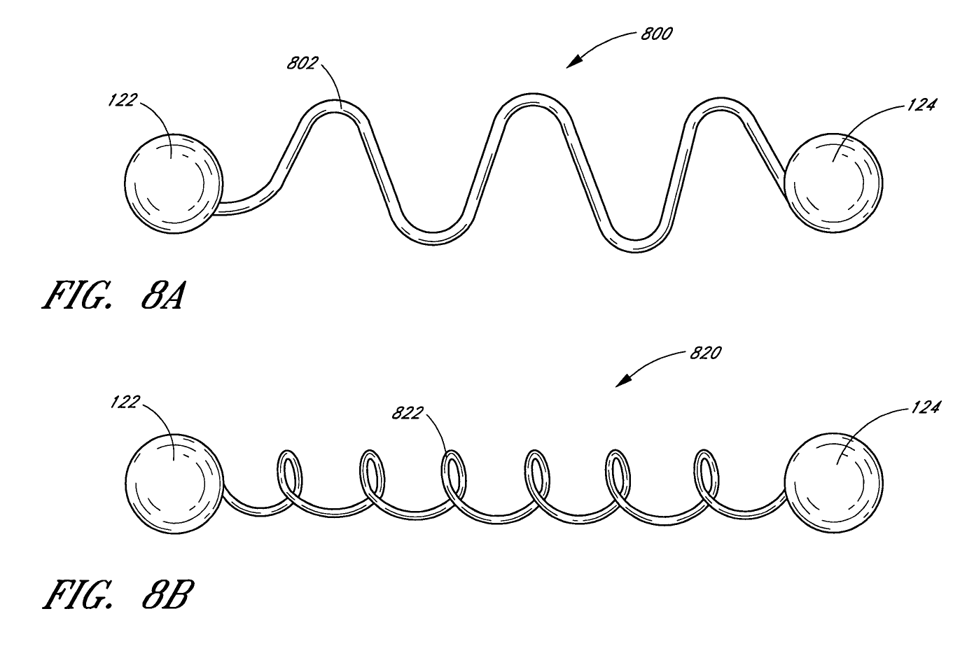

FIG. 6A depicts an embodiment of the invention wherein a tissue shaping device 620 includes a distal end 622 and a proximal end 624 that are coupled to an elongated body 626. The tissue shaping device 620 also includes an outer jacket 628 and an outer layer 630 for facilitating medical procedures using the tissue shaping device 620.

In one embodiment, the outer layer 630 comprises a lubricious material that facilitates placement of the tissue shaping device 620 within the coronary sinus 104. In one embodiment, the lubricious material is hydrogel or TEFLON®. In other embodiments, the lubricious material may comprise surface treated silicone or polyurethane materials, combinations of the same or the like.

In another embodiment of the invention, the outer layer 630 comprises an anti-inflammatory coating to decrease inflammation response by the body of the patient. In one embodiment, the anti-inflammatory coating is Dexamethasone sodium phosphate or Dexamethasone sodium acetate. In other embodiments, the anti-inflammatory coating may comprise heparin or the like.

FIG. 6B illustrates a transverse cross-sectional view of the tissue shaping device 620 of FIG. 6A taken along lines 6B-6B of FIG. 6A. The outer layer 630 is depicted as encircling the outer jacket 628, which encapsulates the elongated body 626. In other embodiments of the invention, either or both of the outer jacket 628 and the outer layer 630 partially enclose, encapsulate, or surround at least one of the ends 622, 624 and/or the body 626.

FIG. 6C illustrates a tissue shaping device 640 according to another embodiment of the invention. In particular, the tissue shaping device 640 includes an outer jacket 642 similar to the outer jacket 128 depicted in FIGS. 1-4. The illustrated outer jacket 642 is substantially adjacent to the ends 622, 624 and to the elongated body 626. That is, a substantial gap does not exist between the elongated body 626 and the outer jacket 642. The tissue shaping device 640 further includes an outer layer 646 similar to the outer layer 630 previously discussed.

FIGS. 7A and 7B illustrate exemplifying tissue shaping devices having passive fixation mechanisms for securing the tissue shaping devices within a vessel, such as the coronary sinus 104. Such passive fixation mechanisms allow for the tissue shaping device to be temporarily or permanently implanted within the subject vessel and prevent the tissue shaping device from undesired movement within the vessel.

FIG. 7A illustrates a tissue shaping device 700 having securing fins for implanting the tissue shaping device 700 within a vessel. As illustrated, the tissue shaping device 700 includes a plurality of distal fins 702 near the distal end 122 of the tissue shaping device 700. The distal fins 702 are shown in a deployed configuration, such as after the tissue shaping device 700 has been positioned within a vessel. In one embodiment, the deployed distal fins 702 have a generally triangular shape and are used to exert pressure against the wall of the subject vessel such that that tissue shaping device 700 is substantially prevented from traveling within the vessel. The tissue shaping device 700 further includes a plurality of proximal fins 704, which are illustrated in an initial, undeployed configuration and are located toward the proximal end 124 of the tissue shaping device 700.

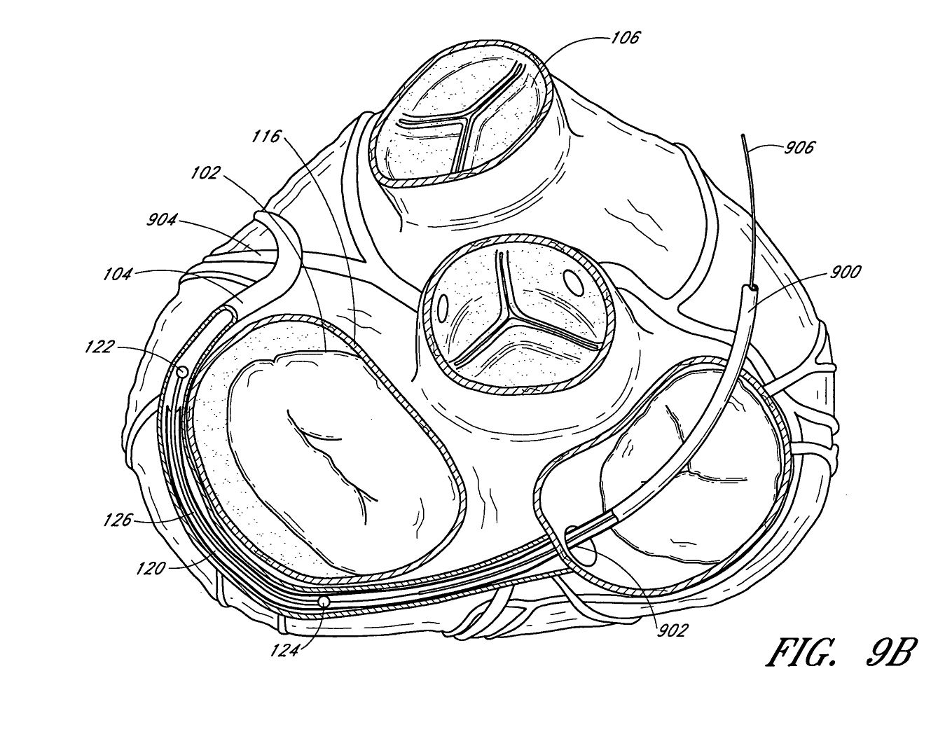

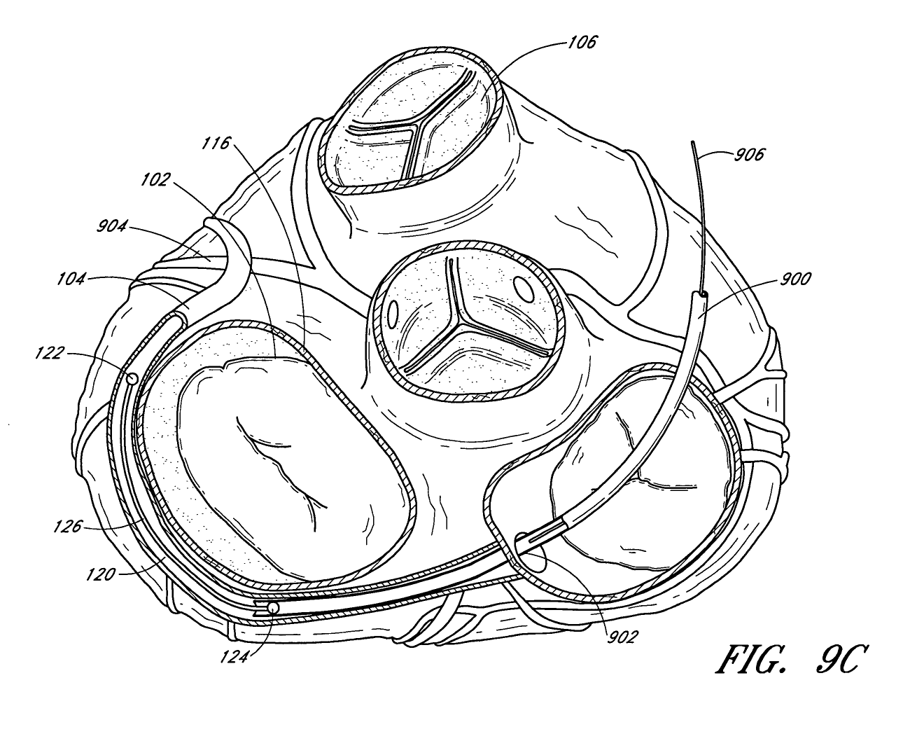

In one embodiment, as the tissue shaping device 700 is being disposed with a vessel, such as through the use of a catheter as described with reference to FIGS. 9A-9C, both the distal fins 702 and the proximal fins 704 are in an initial, undeployed configuration. As the tissue shaping device 700 is withdrawn from or advances out of the catheter, the fins 702, 704 expand to the deployed configuration and substantially secure the tissue shaping device 700 within the vessel.

In one embodiment, the fins 702, 704 are advantageously attached to the outer jacket 128 of the tissue shaping device 700. In yet other embodiments, the fins 702, 704 are incorporated as part of the outer jacket 128. In one embodiment, the fins 702, 704 comprise a flexible material, such as, for example, silicone or polyurethane. In other embodiments, the fins 702, 704 are advantageously constructed of a braided material, such as, for example, stainless steel, nylon or any other suitable combination of metals and/or polymers.

As illustrated, the tissue shaping device 700 comprises two distal fins 702 and two proximal fins 704. In yet other embodiments, other numbers of fins may be used. For example, the distal fins 702 and/or the proximal fins 704 may comprise one fin, three fins, four fins, or more than four fins that are usable to secure the tissue shaping device 700 within a vessel. The plurality of distal fins 702 and/or proximal fins 704 may be different shapes and/or sizes, may be substantially equally spaced around the circumference of the tissue shaping device 700 or may have unequal spacing. In yet other embodiments, the tissue shaping device 700 may have only one set of fins or may include other sets of fins used in addition to the distal fins 702 and proximal fins 704.

FIG. 7B illustrates a tissue shaping device 720 having securing tines for implanting the tissue shaping device 720 within a vessel. As illustrated, the tissue shaping device 720 includes a plurality of distal tines 722 near the distal end 122 of the tissue shaping device 720. The distal tines 722 are shown in a deployed configuration, such as after the tissue shaping device 720 has been disposed within a vessel. In one embodiment, the deployed distal tines 722 have a generally oblong shape and are used to exert pressure against the wall of the subject vessel such that that tissue shaping device 720 is substantially prevented from traveling within the vessel. The tissue shaping device 720 further includes a plurality of proximal tines 724, which are illustrated in an initial, undeployed configuration and are located toward the proximal end 124 of the tissue shaping device 720.

In one embodiment, as the tissue shaping device 720 is being disposed with a vessel, such as through the use of a catheter as described with reference to FIGS. 9A-9C, both the distal tines 722 and the proximal tines 724 are in an initial, undeployed configuration. As the tissue shaping device 720 is withdrawn from or advances out of the catheter, the tines 722, 724 expand to the deployed configuration and substantially secure the tissue shaping device 720 within the vessel.

In one embodiment, the tines 722, 724 are advantageously attached to the outer jacket 128 of the tissue shaping device 720. In yet other embodiments, the tines 722, 724 are incorporated as part of the outer jacket 128. In one embodiment, the tines 722, 724 comprise a substantially flexible material such as, for example, silicone or polyurethane. In other embodiments, the tines 722, 724 are advantageously constructed of a braided material, such as, for example, stainless steel, nylon or any other suitable combination of metals and/or polymers.

As illustrated, the tissue shaping device 720 comprises two distal tines 722 and two proximal tines 724. In yet other embodiments, other numbers of tines may be used. For example, the distal tines 722 and/or the proximal tines 724 may comprise one tine, three tines, four tines, or more than four tines that are usable to secure the tissue shaping device 720 within a vessel. The plurality of distal tines 722 and/or proximal tines 724 may be different shapes and/or sizes, may be substantially equally spaced around the circumference of the tissue shaping device 720 or may have unequal spacing. In yet other embodiments, the tissue shaping device 720 may have only one set of tines or may include other sets of tines used in addition to the distal tines 722 and proximal tines 724.

Although the passive fixation mechanisms are disclosed with reference to particular embodiments, other types of passive fixation mechanisms may be used with embodiments of the present invention. For example, the tissue shaping device may include barbs, bristle-like projections, anchor pads, combinations of the same or the like. In other embodiments of the invention, multiple types of passive fixation mechanisms may be used with the same tissue shaping device. Other types of fixation mechanisms usable with embodiments of the present invention also include active fixation mechanisms, such as, for example, screw-in mechanisms.

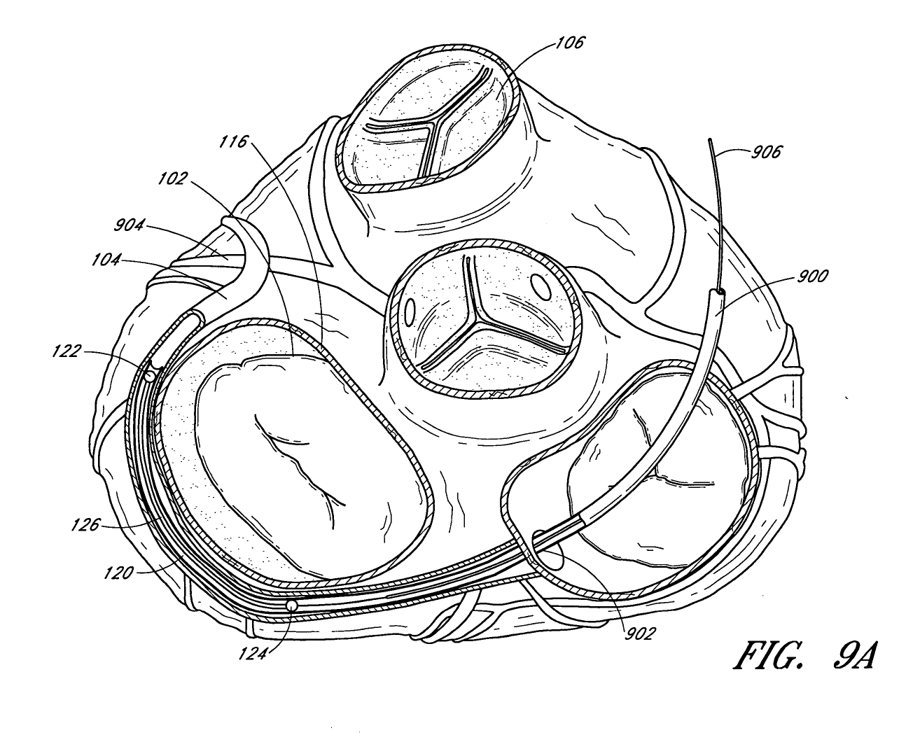

FIGS. 8A and 8B illustrate embodiments of tissue shaping devices having elongated bodies with forms other than a substantially cylindrical rod. FIG. 8A depicts a tissue shaping device 800 having a generally curvilinear-shaped body 802 connected to ends 122, 124. FIG. 8B depicts a tissue shaping device 820 having a generally helical-shaped body 822 connected to ends 122, 124. Both bodies 802 and 822 advantageously provide for enough flexibility to allow for deformation of the bodies 802 and 822 when the ends 122, 124 attract or repel each other due to, for example, magnetic forces. Furthermore, the bodies 802 and 822 provide enough rigidity such that the bodies 802 and 822 do not collapse under forces caused by the attraction of ends 122, 124.

In other embodiments, other forms or shapes of bodies, as discussed above, may be used with the tissue shaping device. Furthermore, in certain embodiments, the tissue shaping devices 800, 820 may include magnetic portions along the length of, or at least partially within, the bodies 802, 822. For example, the curvilinear-shaped body 800 may include at least one magnetic portion on or in a curved portion of the body 800 instead of, or in addition to, at the end of the body 800.

FIGS. 9A-9C depict an exemplary method usable to position the tissue shaping device 120 within the coronary sinus 104. As shown in FIG. 9A, a tubular member, including a catheter 900, is maneuvered into the coronary sinus 104 through the ostium 118. Disposed within the catheter 900 is the tissue shaping device 120 in a first configuration, such that deformation of the tissue shaping device 120 due to attraction of the magnets has not yet fully occurred.

In one embodiment, the catheter 900 is used to position the tissue shaping device 120 distally within the coronary sinus 104 without applying substantial compressive force on a circumflex artery 904 or other major coronary arteries. For example, the distal end of catheter 900 may be disposed at a location proximal to the crossover point between the circumflex artery 904 and the coronary sinus 104, as shown in FIG. 9A. At this point, the catheter 900 is withdrawn proximally while the tissue shaping device 120 is held stationary, such as by a control wire 906, to uncover the tissue shaping device 120 within the coronary sinus 104, as is depicted in FIGS. 9B and 9C. Alternatively, the catheter 900 may be held stationary while the tissue shaping device 120 is advanced out of the distal end of the catheter 900. In yet other embodiments, other methods known to those skilled in the art may be used to deploy the tissue shaping device 120 within the coronary sinus 104 or other subject vessel or location within the patient's body.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A method for changing a dimension of a mitral valve annulus of a heart, the method comprising:
positioning an implant at least partially in a coronary sinus of the heart, the implant comprising a first magnetic portion and a second portion, the second portion being responsive to a magnetic field emanating from the first magnetic portion, wherein the first magnetic portion and the second portion are configured to change the implant from a first configuration to a second configuration, said second configuration producing a change in the dimension of the mitral valve annulus.

2. The method of claim 1, wherein the first magnetic portion and second portion are located at or hear respective ends of said implant.

3. The method of claim 1, wherein the change in the dimension comprises a decrease.

4. The method of claim 3, wherein at least one force exerted upon the coronary sinus by the implant in said second configuration produces said decrease in the dimension.

5. The method of claim 1, further comprising delivering the implant to the coronary sinus with an elongate tubular body, wherein the implant is in said first configuration while located in or on the tubular body.

6. The method of claim 5, wherein the implant assumes said second configuration during or after being released from the tubular body into the coronary sinus.

7. The method of claim 1, further comprising electrically activating at least one of the first magnetic portion and the second portion.

8. The method of claim 7, wherein said electrically activating comprises activating at least of the first magnetic portion and the second portion with an electromagnetic transmitter located outside the heart.

9. The method of claim 1, further comprising positioning a second implant within the coronary sinus of the heart.

10. A tissue shaping device comprising:
an elongate, flexible body configured to fit within a coronary sinus of a heart;
a first magnetic portion located in or on the body; and
a second portion located in or on the body, the second portion configured to respond to a magnetic field emanating from the first magnetic portion,
wherein the first magnetic portion is configured to interact with the second portion such that the body changes shape from a first configuration to a second configuration.

11. The tissue shaping device of claim 10, wherein the second portion is magnetic.

12. The tissue shaping device of claim 11, wherein said interaction between the first magnetic portion and the second portion is an attraction.

13. The tissue shaping device of claim 10, wherein the device is configured such that, when the body is in said second configuration, the device exerts at least one force sufficient to decrease a dimension of a mitral valve annulus when the device is positioned within the coronary sinus.

14. The tissue shaping device of claim 10, wherein the first magnetic portion comprises a rare earth element.

15. The tissue shaping device of claim 14, wherein the first magnetic portion comprises at least one of the following: NdFeB (Neodymium Iron Boron), SmCo (Samarium Cobalt) and AlNiCo (Aluminum Nickel Cobalt).

16. The tissue shaping device of claim 10, wherein the body comprises a curvilinear shape.

17. The tissue shaping device of claim 10, further comprising a jacket that at least partially surrounds the first magnetic portion.

18. The tissue shaping device of claim 10, further comprising at least one fixation member configured to substantially anchor the device within the coronary sinus.

19. The tissue shaping device of claim 10, wherein said second configuration comprises a substantially arcuate shape.

20. A device for reshaping or reforming body tissue, the device comprising:
means for emanating a magnetic field;
means for interacting with the means for emanating by responding to the magnetic field; and
elongate, flexible means coupled to the means for emanating and the means for interacting, the elongate, flexible means configured to fit within a coronary sinus of a heart, and wherein the elongate, flexible means changes from a first configuration to a second configuration while the means for interacting responds to the magnetic field.
